(19) United States
(12) Patent Application Publication
(10) Pub. No.: US 2024/0285151 A1
(43) Pub. Date: Aug. 29, 2024

(54) MEDICAL SYSTEMS, DEVICES, AND RELATED METHODS FOR ROTATABLY OR PIVOTABLY COUPLING MEDICAL DEVICES

(71) Applicant: Boston Scientific Scimed, Inc., Maple Grove, MN (US)

(72) Inventors: Alejandro GUILARTE, Marlborough, MA (US); John Thomas FAVREAU, Charlton, MA (US); Douglas MELANSON, Natick, MA (US); Jeffrey V. BEAN, Fitchburg, MA (US)

(73) Assignee: Boston Scientific Scimed, Inc., Maple Grove, MN (US)

(21) Appl. No.: 18/588,336

(22) Filed: Feb. 27, 2024

Related U.S. Application Data
(60) Provisional application No. 63/487,283, filed on Feb. 28, 2023.

(51) Int. Cl. A61B 1/00

(52) U.S. Cl. CPC A61B 1/00128

(57) ABSTRACT

A coupling device for a medical system includes a first portion and a second portion. The first portion includes a first body and a first connector. The first connector extends from the first body and is configured to couple a first medical device to the first portion. The second portion includes a second body and a second connector. The second connector extends from the second body and is configured to couple a second medical device to the second portion. The first portion and the second portion are removably couplable, and, when coupled, the second portion are rotatable relative to the first portion.

### CROSS-REFERENCE TO RELATED APPLICATIONS

This application claims the benefit of priority to U.S. Provisional Application No. 63/487,283, filed on Feb. 28, 2023, which is incorporated by reference herein in its entirety.

### TECHNICAL FIELD

This disclosure generally relates to medical systems, devices, and related methods that may be used to treat a subject. Aspects of the disclosure relate to medical systems, devices, and methods for rotatably or pivotably coupling medical devices, for example, for endoscopic medical procedures, such as cutting, capturing, or otherwise manipulating tissue, objects, or material with one or more medical devices, among other aspects.

### BACKGROUND

Various medical techniques and instruments have been developed for diagnosis and/or treatment within a patient's body, such as within a patient's gastrointestinal (GI) tract or within a patient's urinary system. Endoscopic sub-mucosal dissection (ESD), endoscopic sub-mucosal resection (ESR), mucosal resection (EMR), polypectomy, mucosectomy, etc., are minimally invasive treatment methods for both malignant and non-malignant lesions. Endoscopic medical procedures, such as, for example, ESR, may be used to excise sessile adenomas or other unwanted tissue (e.g., tumors attached to a bodily surface) from the surface of an anatomical lumen (e.g., stomach, esophagus, colon, etc.). Such procedures often require multiple endoscopic instruments for the resection of tissue. In some instances, a secondary medical device may be coupled to and/or inserted through a portion of a primary medical device in order allow one user to hold and/or manipulate both the primary medical device and the secondary medical device, for example, to treat a treatment site. However, coupling the secondary medical device to the primary medical device and/or inserting the secondary medical device through the primary device may limit the movement of the secondary medical device relative to primary medical device, which may increase user fatigue, impede the manipulation and/or articulation of one or more of the primary medical device and/or the second medical device, or otherwise affect the procedure.

These concerns may increase the duration, costs, and risks of the medical procedure. The systems, devices, and methods of this disclosure may rectify some of the deficiencies described above or address other aspects of the art.

### SUMMARY

Examples of the disclosure relate to, among other things, systems, devices, and methods for performing one or more medical procedures, for example, endoscopic medical procedures, such as cutting, capturing, or otherwise manipulating tissue, objects, or material with one or more medical devices. Each of the examples disclosed herein may include one or more of the features described in connection with any of the other disclosed examples.

In some aspects, a coupling device for a medical system may include a first portion and a second portion. The first portion may include a first body and a first connector. The first connector may extend from the first body and may be configured to couple a first medical device to the first portion. The second portion may include a second body and a second connector. The second connector may extend from the second body and may be configured to couple a second medical device to the second portion. The first portion and the second portion may be removably couplable, and, when coupled, the second portion may be rotatable relative to the first portion.

The coupling device may include one or more of the following features. The first body may include a first coupling projection. The second body may include a second coupling projection configured to be coupled to the first coupling projection. The first coupling projection may include a socket, and the second coupling projection may include a ball.

The first body may include a first magnet, and the second body may include a second magnet of opposite polarity to the first magnet. The second body may include a plurality of second protrusions configured to abut and rotate relative to the first protrusions. Each of the first protrusions and the second protrusions may include a trapezoidal shape with angled side surfaces. The first body further may include a circular depression, and the first protrusions may be positioned within the circular depression. The first body may include a plurality of indentations positioned in a circular arrangement around the first magnet, and the second body may include a plurality of protrusions in a circular arrangement around the second magnet. The indentations may be generally circular. The protrusions may be generally circular. The first body further may include a circular indentation that at least partially overlaps with the indentations, and the second body further may include a circular protrusion that at least partially overlaps with the protrusions.

The first body may include a ratchet and a pawl. The ratchet may include a center hole, and the second body may include a peg configured to positioned within the center hole to couple the second body to the first body. The first body may include a first material, and the second body may include a second material that is releasably couplable to the first material. The first body may include a protrusion, and the second body may include an indentation configured to key with and be coupled to the protrusion via a press fit. The first body may include a first coupling projection with first and second extensions that each include respective sides openings. The second body may include a second coupling projection with a central prong, a first side prong, and a second side prong. The first side prong and the second side prong may be both pivotable inward toward the central prong to couple or uncouple the first body and the second body. The first body may include a first receiving portion with a first slot, and the first connector may include a first narrowed portion configured to be positioned within the first slot. The second body may include a second receiving portion with a second slot, and the second connector may include a second narrowed portion configured to be positioned within the second slot.

In another aspect, a medical system may include a first medical device, a second medical device, and a coupling device. The first medical device may include a first handle and a first insertion portion, and the first handle may include a port. The second medical device may include a second handle and a second insertion portion. At least a portion of the second insertion portion may be configured to be inserted into the port. The coupling device may include a first portion and a second portion. The first portion may include a first body, and the first body may include a first receiving portion with a first slot. The first slot may be configured to receive a portion of a first strap to couple the first handle to the first portion. The second portion may include a second body, and the second body may include a second receiving portion with a second slot. The second slot may be configured to receive a portion of a second strap to couple the second handle to the second portion. The first portion and the second portion may be removably couplable, and, when coupled, the second portion is rotatable relative to the first portion such that the second handle.

The medical system may include one or more of the following aspects. The first body may include a first coupling projection, and the second body may include a second coupling projection configured to be coupled to the first coupling projection. The first coupling projection may include a socket, and the second coupling projection may include a ball. The first body may include a first magnet, and the second body may include a second magnet of opposite polarity to the first magnet. The first body may include a plurality of first protrusions, and the second body may include a plurality of second protrusions configured to abut and rotate relative to the first protrusions. Each of the first protrusions and the second protrusions may include a trapezoidal shape with angled side surfaces.

In a further aspect, a coupling device may include a first portion and a second portion. The first portion may include a first body and a first strap. The first strap may extend from the first body and may be configured to couple a first medical device to the first portion. The first portion may include a socket extending from the first body. The second portion may include a second body and a second strap. The second strap may extend from the second body and may be configured to couple a second medical device to the second portion. The second portion may include a hollow ball configured to be positioned within the socket of the first portion. The first portion and the second portion may be removably couplable, and, when coupled, the second portion may be pivotable relative to the first portion through a semi-spherical range of motion.

The coupling device may include one or more of the following features. The first body may include a first receiving portion with a first slot. The first strap may include a first narrowed portion configured to be positioned within the first slot. The second body may include a second receiving portion with a second slot. The second strap may include a second narrowed portion configured to be positioned within the second slot.

It may be understood that both the foregoing general description and the following detailed description are exemplary and explanatory only and are not restrictive of this disclosure, as claimed.

### BRIEF DESCRIPTION OF THE DRAWINGS

The accompanying drawings, which are incorporated in and constitute a part of this specification, illustrate exemplary aspects of the disclosure and together with the description, serve to explain the principles of the disclosure.

FIGS. 1A-1E illustrate a medical system, including a primary medical device, a secondary medical device, and a coupling device, in various configurations, according to aspects of this disclosure.

FIGS. 2A-2E illustrate another medical system, including the primary medical device, the secondary medical device, and another coupling device, in various configurations, according to aspects of this disclosure.

FIGS. 3A and 3B are perspective views of another coupling device in an uncoupled configuration, according to aspects of the disclosure.

FIGS. 4A and 4B are perspective views of yet another coupling device in an uncoupled configuration, according to aspects of the disclosure.

FIGS. 5A and 5B are perspective views of a further coupling device in an uncoupled configuration, according to aspects of the disclosure.

FIGS. 6A and 6B are perspective views of yet a further coupling device in an uncoupled configuration, according to aspects of the disclosure.

FIGS. 7A and 7B are perspective views of another coupling device in an uncoupled configuration, according to aspects of the disclosure.

FIGS. 8A and 8B are side views of a further coupling device in an uncoupled configuration and in a coupled configuration, respectively, according to aspects of the disclosure.

### DETAILED DESCRIPTION

The terms “proximal” and “distal” are used herein to refer to the relative positions of the components of an exemplary medical system and exemplary medical devices. When used herein, “proximal” refers to a position relatively closer to the exterior of the body of a subject or closer to a medical professional using the medical system or medical device. In contrast, “distal” refers to a position relatively further away from the medical professional using the medical system or medical device, or closer to the interior of the body of the subject. Proximal and distal directions are labeled with arrows marked “P” and “D”, respectively, throughout various figures. As used herein, the terms “comprises,” “comprising,” “having,” “including,” or other variations thereof, are intended to cover a non-exclusive inclusion, such that a system, device, or method that comprises a list of elements does not include only those elements, but may include other elements not expressly listed or inherent thereto. Unless stated otherwise, the term “exemplary” is used in the sense of “example” rather than “ideal.” As used herein, the terms “about,” “substantially,” and “approximately,” indicate a range of values within +/−10% of a stated value.

Embodiments of this disclosure include devices, systems, and methods for manipulating, cutting, grabbing, ligating, and/or otherwise treating tissue. In some examples, the devices, systems and/or methods discussed herein may be utilized during endoscopic mucosal resection (EMR) and/or endoscopic submucosal dissection (ESD) procedures. In examples, EMR and ESD include endoluminal placement of one or more devices for grasping and cutting tissue proximate to a target area within the body of a patient. Placement of the one or more medical devices may be via a catheter, scope (endoscope, bronchoscope, colonoscope, gastroscope, duodenoscope, etc.), tube, or sheath, inserted into the GI tract via a natural orifice or incision. The orifice can be, for example, the nose, mouth, or anus, and the placement can be in any portion of the GI tract, including the esophagus, stomach, duodenum, large intestine, or small intestine. Placement also can be in other organs reachable via the GI tract. In other aspects, the one or more medical devices may be delivered via a catheter, scope (e.g., ureteroscope), tube, or sheath, inserted into the urinary system via a natural orifice (e.g., urethra) or incision. The patient's tissue may be grasped using suction from one or more medical devices and/or a grasper, and then the tissue may be cut by a cutting device for subsequent removal from the patient's body. In examples, the devices, systems and/or methods discussed herein may be utilized during natural orifice transluminal endoscopic surgery (NOTES) procedures. In a NOTES procedure, a stent or other device may be delivered in order to create an opening between two body lumens (e.g., a stomach and a small bowel). Although EMR, ESD, and NOTES are discussed herein, the disclosure is not so limited. Embodiments of the disclosure include systems and devices that may be used (and methods that may be performed) in any suitable procedure in any body lumen or organ.

Reference will now be made in detail to examples of this disclosure described above and illustrated in the accompanying drawings. Wherever possible, the same reference numbers will be used throughout the drawings to refer to the same or like parts.

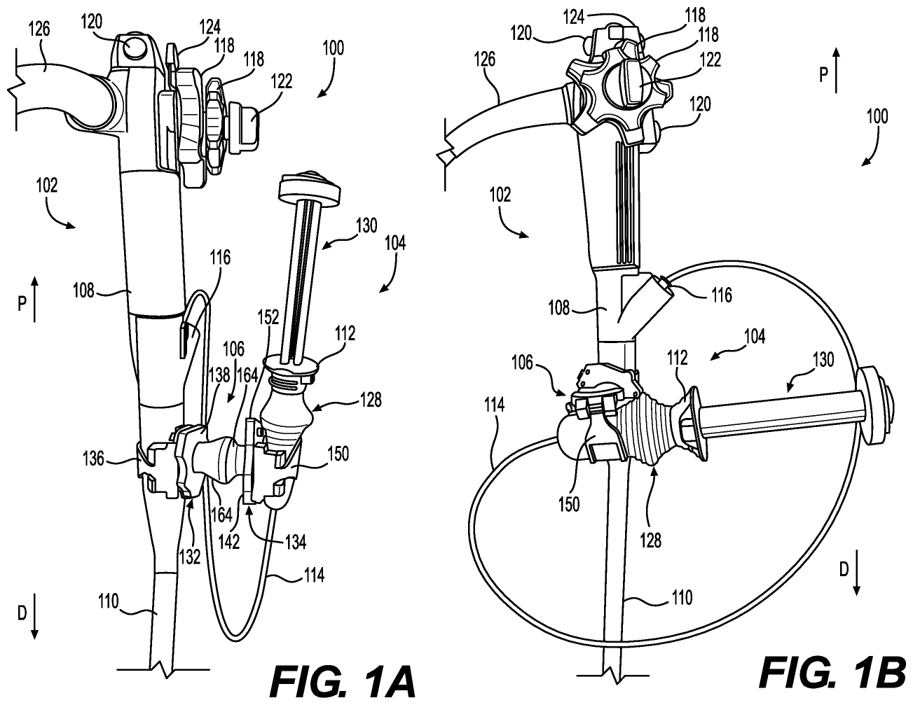

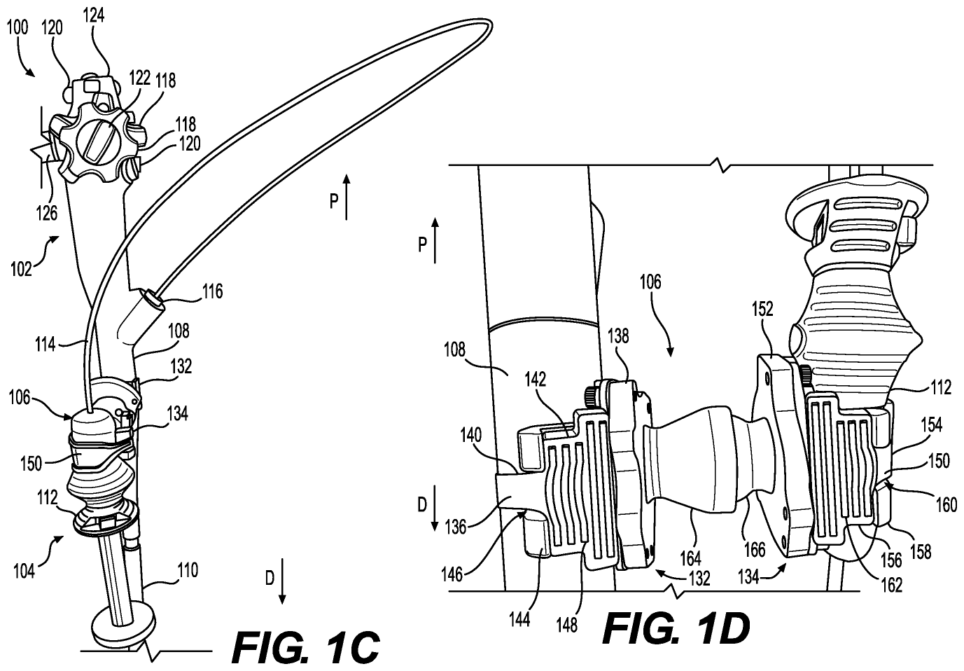

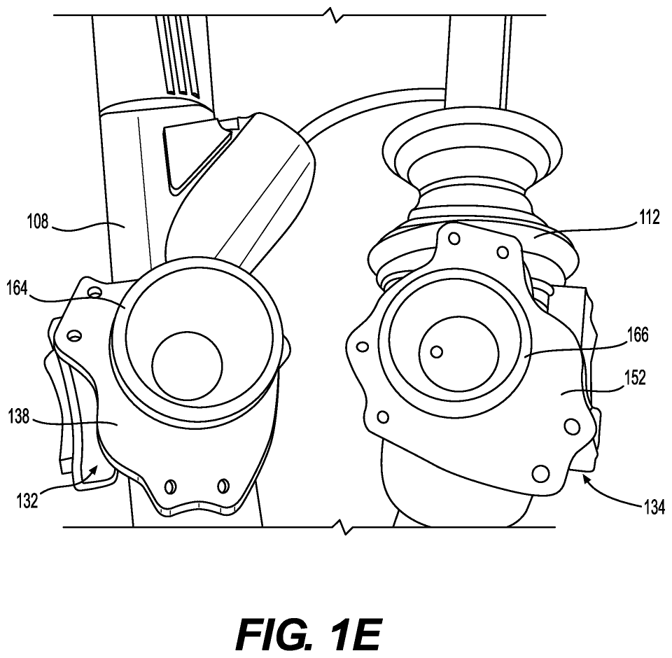

FIGS. 1A-1E illustrate various views of an exemplary medical system 100, including a first medical device 102 and a second medical device 104. First medical device 102 and second medical device 104 may be coupled (e.g., releasably coupled) via a coupling device 106. First medical device 102 includes a handle 108 and an insertion portion 110, and second medical device 104 includes a handle 112 and an insertion portion 114. In some aspects, handle 112 of second medical device 104 may be coupled to handle 108 of first medical device 104 via coupling device 106. Moreover, insertion portion 110 of first medical device 102 may be inserted into a subject, and insertion portion 114 of second medical device 104 may be delivered through a portion of insertion portion 110, for example, via a port 116 or other opening in handle 108 of first medical device 102. As discussed in detail below, coupling device 106 may allow for handle 112 of second medical device 104 to be movable (e.g., pivotable, rotatable, etc.) relative to handle 108 of first medical device 102. Moreover, coupling device 106 may allow for handle 112 of second medical device 104 to be coupled to and uncoupled from (e.g., fully detached and separated from) handle 108 of first medical device 102.

First medical device 102 includes handle 108 and insertion portion 110, which includes a distal end (not shown). One or more lumens may extend from handle 108 to the distal end, for example, through insertion portion 110. As mentioned, handle 108 includes port 116 or another opening, which may connect through handle 108 to the one or more lumens in insertion portion 110. Moreover, handle 108 may include one or more knobs 118 and one or more actuators 120. For example, knobs 118 may be movable (rotatable or pivotable) to control the movement (e.g., deflection) of a distal portion of insertion portion 110. Handle 108 may also include one or more locking devices 122, for example, to secure the position of knobs 118, and thus secure the position of the distal portion of insertion portion 110. Additionally, actuators 120 may include one or more buttons or other types of actuators. Actuators 120 may be controlled to actuate the delivery of fluid, to actuate the application of suction, to actuate one or more illumination or imaging devices, etc. Furthermore, handle 108 may include one or more elevator actuator 124, for example, to control the position of an elevator positioned within the lumen of insertion portion 110.

First medical device 102 may include or be coupled to an umbilicus 126, for example, coupled to a proximal portion of handle 108. Umbilicus 126 may connect first medical device 102 to one or more of a control unit, a fluid source, a suction source, and/or other exterior devices, such as a monitor for viewing images from a camera.

Second medical device 104 includes handle 112 and insertion portion 114, and insertion portion 114 may be inserted through port 116. Additionally, handle 112 of second medical device 104 may include a stationary portion 128 and a movable portion 130. In these aspects, movement of movable portion 130 relative to stationary portion 128 may control one or more aspects of second medical device 104. For example, second medical device 104 may include one or more end effectors, and movement of movable portion 130 may control the position, extension, configuration, etc. of the one or more end effectors. Additionally or alternatively, handle 112 may include controls similar to knob(s) 118, actuator(s) 120, locking device(s) 122, or elevator actuator(s) 124, and insertion portion 114 may define one or more lumens for receiving other medical devices.

Coupling device 106 includes a first portion 132 and a second portion 134. First portion 132 may be couplable to first medical device 102, and second portion 134 may be couplable to second medical device 104. For example, as shown in FIG. 1A-1D, first portion 132 may include a first connector or strap 136 and first body 138. First strap 136 may be configured to wrap around a portion of handle 108. For example, one end of first strap 136 may be fixedly coupled to a portion of first body 138. As shown in more detail in FIG. 1D, another end of first strap 136 may include a narrowed portion 140 and a widened portion 142, for example, with the widened portion 142 at the another end. Additionally, a portion of first body 138 may include a receiving portion 144, for example, including a slot 146. In these aspects, the narrowed portion 140 of first strap 136 may be positioned within slot 146 of receiving portion 144 to couple (e.g., removably couple) first body 138 to handle 108. Additionally, widened portion 142 may help to securely couple first strap 136 to first body 138. In some aspects, as shown in FIG. 1D, one or more portions of widened portion 142 may include raised and/or depressed sections, for example, forming a grip surface 148. First strap 136 may thus be utilized to removably couple first portion 132 to first medical device 102. Alternatively, first portion 132 may be fixedly coupled to first medical device 102 (e.g., integrated into handle 108).

Second portion 134 may be couplable to second medical device 104. As shown in FIGS. 1A-1D, second portion 134 may include a second connector or strap 150 and second body 152. Second strap 150 may be configured to wrap around a portion of handle 112. For example, one end of second strap 150 may be fixedly coupled to a portion of second body 152. Another end of second strap 150 may include a narrowed portion 154 and a widened portion 156, for example, with the widened portion 156 at the another end. Additionally, a portion of first body 152 may include a receiving portion 158, for example, including a slot 160. In these aspects, the narrowed portion 154 of second strap 150 may be positioned within slot 160 of receiving portion 158 to couple (e.g., removably couple) second body 152 to handle 112. Additionally, widened portion 156 may help to securely couple second strap 150 to second body 152. In some aspects, as shown in FIG. 1D, one or more portions of widened portion 156 may include raised and/or depressed sections, for example, forming a grip surface 162. Second strap 150 may thus be utilized to removably couple second portion 134 to second medical device 104. Alternatively, second portion 134 may be fixedly coupled to second medical device 104 (e.g., integrated into handle 112).

As described above, first portion 132 may be coupled to first medical device 102 and second portion 134 may be coupled to second medical device 104 using a same type of attachment mechanism (e.g., a strap, such as first strap 136 and second strap 150). Alternatively, first portion 132 and second portion 134 may utilize alternative attachment mechanisms, and the attachment mechanisms used for first portion 132 and second portion 134 may differ from one another and may include mechanisms such as jaws, clips, ratchets, or other mechanisms.

Moreover, first portion 132 includes a first coupling projection 164, and second portion 134 includes a second coupling projection 166. In some aspects, for example, as shown in FIGS. 1A and 1D, first coupling projection 164 may form a socket, and second coupling projection 166 may form a ball. Alternatively, first coupling projection 164 may form a ball, and second coupling projection 166 may form a socket. In either aspect, as shown in FIG. 1E, the ball may be hollow, for example, sized to pivotably fit within the socket, but hollow on the inside. For example, the ball may have a bowl-like shape. Additionally, in either aspect, first portion 132 and second portion 134 may releasably couple first medical device 102 and second medical device 104 (e.g., via respective handles). As such, first medical device 102 and/or second medical device 104 may be rotated or otherwise repositioned relative to the other of first medical device 102 and/or second medical device 104, while helping to ensure that first medical device 102 and second medical device remain coupled together. Accordingly, as shown in FIGS. 1A-1E, coupling device 106 allows for handle 112 of second medical device 104 to be rotated, for example, through approximately a semi-spherical range of motion, relative to handle 108 of first medical device 102.

Moreover, first medical device 102 may be selectively coupled to coupling device 106, for example, via first portion 132 and first strap 136. Similarly, second medical device 104 may be selectively coupled to coupling device 106, for example, via second portion 134 and second strap 150. Furthermore, first coupling projection 164 and second coupling projection 166 may be uncoupled (e.g., by removing the ball from the socket). Uncoupling first coupling projection 164 and second coupling projection 166 may allow the user (or another user) to operate second medical device 104 at a location separated from first medical device 102, replace second medical device 104 with a new medical device (e.g., with a new second portion 134), or otherwise provide flexibility in the usage of medical system 100.

In some aspects, first medical device 102 may be a gastroscope. Alternatively, first medical device 102 may be one of other similar insertion devices with a handle, such as, for example, endoscopes, ureteroscopes, duodenoscopes, endoscopic ultrasonography (“EUS”) scopes, colonoscopes, bronchoscopes, laparoscopes, arthroscopes, cystoscopes, aspiration scopes, etc. Moreover, second medical device 104 may be a stent delivery device, for example, with a stent releasably positioned at a distal end of insertion portion 114, such that manipulation of handle 112 (e.g., of movable portion 130) controls the deployment, delivery, or positioning of the stent. Alternatively, second medical device 104 may be one of other end effector devices, such as, for example, graspers, clamps, clips, electrosurgical knives or other electrocautery devices, retrieval device, hemostatic patches, etc. Second medical device alternatively may be a scope, such as a cholangioscope.

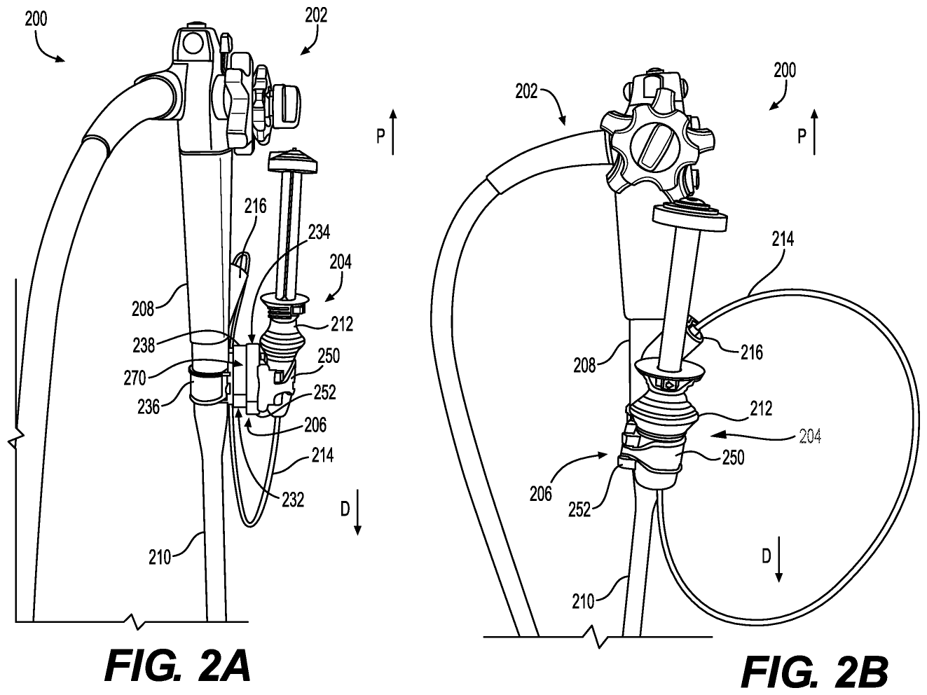

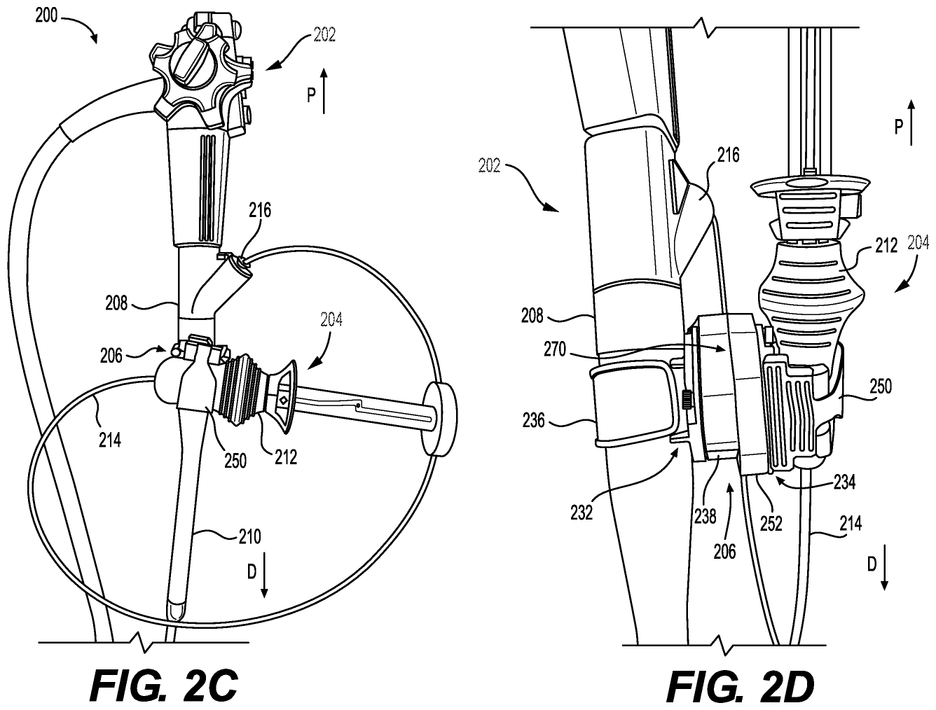

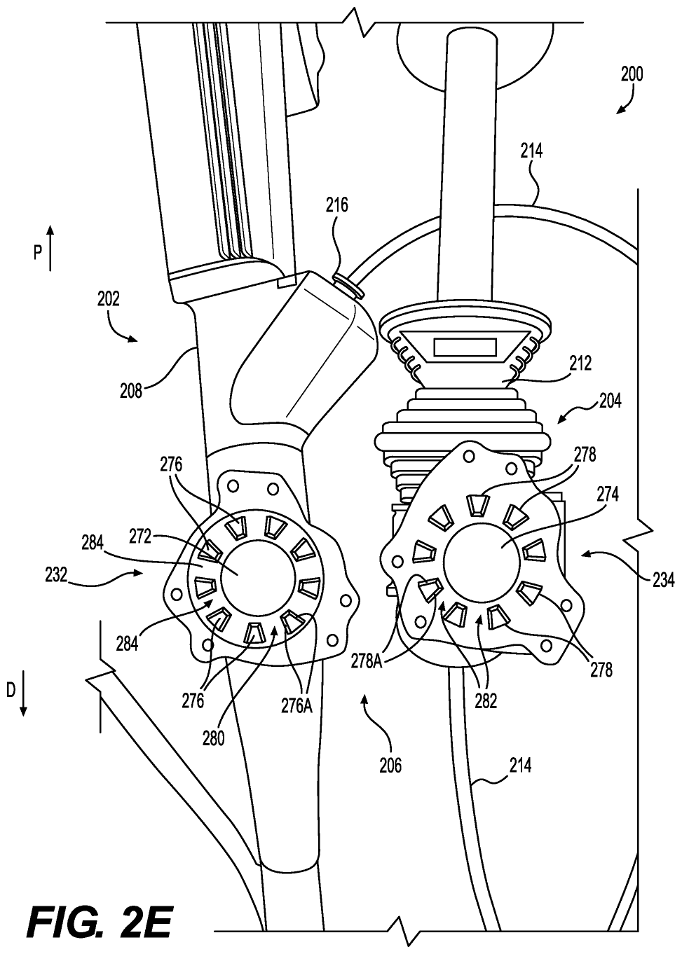

FIGS. 2A-2E illustrate another exemplary medical system 200, for example, including a first medical device 202, a second medical device 204, and a coupling device 206. First medical device 202 includes a handle 208 and an insertion portion 210, and second medical device 204 includes a handle 212 and an insertion portion 214. As discussed herein, coupling device 206 may selectively couple handle 208 of first medical device 202 and handle 212 of second medical device 204. Moreover, insertion portion 214 of second medical device 204 may be delivered through insertion portion 210 of first medical device 202, for example, via a port 216. Additionally, first medical device 202 and second medical device 204 may include one or more of the features discussed above with respect to first medical device 102 and second medical device 104 in FIGS. 1A-1E.

Coupling device 206 includes a first portion 232 and a second portion 234. As discussed above, first portion 232 may include a first connector or strap 236 and a first body 238, such that first strap 236 may wrap around first medical device 202 (e.g., around handle 208) and be secured to first body 238 to couple first portion 232 to first medical device 202. Moreover, second portion 234 may include a second connector or strap 250 and a second body 252, such that second strap 250 may wrap around second medical device 204 (e.g., around handle 212) and be secured to second body 252 to couple second portion 234 to second medical device 204.

As shown in FIGS. 2A and 2D, first portion 232 and second portion 234 may at least partially abut, for example, at a junction 270. As shown in FIGS. 2A-2D, first portion 232 and second portion 234 may releasably couple first medical device 202 and second medical device 204 (e.g., via respective handles). As such, first medical device 202 and/or second medical device 204 may be rotated or otherwise repositioned relative to the other of first medical device 202 and/or second medical device 104, while helping to ensure that first medical device 202 and second medical device remain coupled together. Accordingly, as shown in FIGS. 2A-2D, coupling device 206 allows for handle 212 of second medical device 204 to be rotated, for example, through approximately a circular range of motion, relative to handle 208 of first medical device 202. As discussed below, coupling device 206 may allow for the positioning of handle 212 relative to handle 208 to be at least partially or temporarily secured in various positions. Moreover, first medical device 202 may be selectively coupled to coupling device 206, for example, via first portion 232 and first strap 236 (see FIGS. 2A and 2D). Similarly, second medical device 204 may be selectively coupled to coupling device 206, for example, via second portion 234 and second strap 250. Furthermore, as discussed below, first medical device 202 and first portion 232 may be separated from second medical device 204 and second portion 234, for example, by uncoupling first portion 232 and second portion 234 at junction 270.

FIG. 2E illustrates first portion 232 and second portion 234 of coupling device 206 separated, such that first medical device 202 and second medical device 204 are separated. Specifically, FIG. 2E illustrates respective portions of first portion 232 and second portion 234 that abut (e.g., at junction 270, FIGS. 2A and 2D) and interact to allow for relative motion of handle 212 relative to handle 208. In some aspects, first portion 232 may include a first magnet 272, and second portion 234 may include a second magnet 274. In these aspects, first magnet 272 and second magnet 274 are of opposite polarity. Accordingly, magnets 272, 274 may help to maintain a connection between first portion 232 and second portion 234, while also allowing for a user to separate first portion 232 and second portion 234 by pulling first portion 232 and second portion 234 apart with sufficient force to overcome the magnetic attractive force of magnets 272, 274.

Moreover, as shown in FIG. 2E, first portion 232 may include a plurality of first protrusions 276, for example, arranged at least partially circularly/circumferentially around first magnet 272. Additionally, second portion 234 may include a plurality of second protrusions 278, for example, arranged at least partially circularly/circumferentially around second magnet 274. As shown in FIG. 2E, each of first protrusions 276 and second protrusions 278 may be generally trapezoidal, for example, with side surfaces 276A, 278A narrowing in a direction toward first magnet 272 or second magnet 274. Additionally, side surfaces 276A and 278A may be angled, for example, such that top portions further from handle 208/handle 212 (as shown in FIG. 2E) of first protrusions 276 and second protrusions 278 are narrower than bottom portions closer to handle 208/handle 212 of the respective one of first protrusions 276 and second protrusions 278. First protrusions 276 may be spaced apart by first gaps 280, and second protrusions may be spaced apart by second gaps 282. In these aspects, when first portion 232 and second portion 234 are coupled together (e.g., as shown in FIGS. 2A-2D), first protrusions 276 may be received within second gaps 282, and second protrusions 278 may be received within first gaps 280, which may help secure the position of handle 212 relative to handle 208.

In some aspects, first portion 232 may include a depression 284, for example, a circular depression surrounding first magnet 272. In other words, first magnet 272 may protrude outward relative to depression 284. First protrusions 276 and first gaps 280 may be positioned within depression 284, such that first portion 232 and second portion 234 may abut at junction 270 when first protrusions 276 are positioned within second gaps 282, and when second protrusions 278 are positioned within first gaps 280. Alternatively or additionally, although not shown, second protrusions 278 and second gaps 282 may arranged in a depression. Alternatively, second magnet 274 may be depressed relative to second gaps 282.

As shown, first portion 232 may include nine first protrusions 276 and nine first gaps 280, and second portion 234 may include nine second protrusions 278 and nine second gaps 282. Alternatively, first portion 232 and second portion 234 may include fewer or more protrusions and gaps. In some aspects, first portion 232 and second portion 234 include the same number of protrusions and gaps. In other aspects, first portion 232 and second portion 234 include different numbers of protrusions and gaps.

Moreover, angled side surfaces 276A and 278A may help to allow for the user to rotate handle 212 relative to handle 208. For example, the user may rotate handle 212 and/or second portion 234 clockwise or counterclockwise, and the abutting side surfaces 276A, 278A of respective projections 276, 278 may guide or ramp second portion 234 away from first portion 232, which may help to allow for rotation of second portion 234. For example, as handle 212 and/or second portion 234 are rotated, projections 278 may be rotate relative to projections 276. As such, one side of each projection 278 may abut one side of a respective projection 276. The respective angled side surface 276A, 278A may ramp or guide projection 278 away from projection 276, pushing second portion 234 away from first portion 232 (e.g., laterally relative to handle 208). First magnet 272 and second magnet 274 may help to ensure second portion 234 remains close or in proximity to first portion 232, even if second portion 234 is not in abutting contact with first portion 232. Moreover, once second portion 234 has been rotated such that projections 276, 278 are positioned in respective gaps 280, 282, first magnet 272 and second magnet 274 may help to retain second portion 234 in abutting contact with first portion 232. First magnet 272 and second magnet 274 may also help to retain the position and/or orientation of second portion 234 relative to first portion 232, and thus also help to retain the position and/or orientation of handle 212 relative to handle 208.

Alternatively, although not shown, one of first portion 232 or second portion 234 may include projections, and the other of first portion 232 or second portion 234 may include indentations, for example, of a complementary shape and size of the projections. In this aspect, both the projections and the indentations may include generally trapezoidal shapes, for example, with angled side surfaces, as discussed above. The projections and indentations may help to secure the position of second portion 234 relative to first portion 232, while also helping to allow for relative rotation.

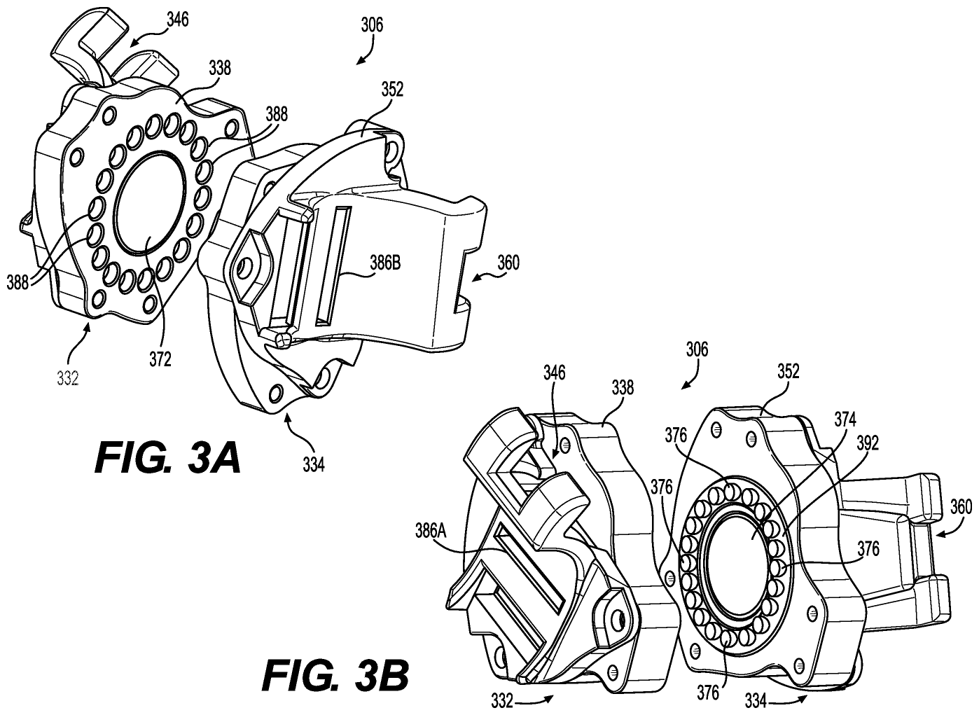

FIGS. 3A and 3B illustrate aspects of an alternative coupling device 306, for example, to rotatably couple a first medical device handle and a second medical device handle. As such, coupling device 306 include a first portion 332 and a second portion 334. First portion 332 may include a first body 338, and second portion 334 may include a second body 352. Although not shown, coupling device 306 may include a first connector or strap and a second connector or strap (or alternative attachment mechanism), as discussed above. For example, as shown in FIG. 3A, second portion 334 may include a second coupling slot 386B to receive a portion of the second strap and a slot 360 to releasably couple second portion 334 to a second medical device. Furthermore, as shown in FIG. 3B, first portion 332 may include a first coupling slot 386A to receive a portion of the first strap and a slot 346 to releasably couple first portion 332 to a first medical device.

Moreover, as shown in FIG. 3A, first portion 332 may include a first magnet 372 and a plurality of indentations 388. Indentations 388 may be positioned in a circular/circumferential arrangement, for example, around (e.g., spaced apart from) first magnet 372. In some aspects, as shown, indentations 388 are evenly spaced around first magnet 372. In other aspects, indentations 388 may be unevenly spaced around first magnet 372. Moreover, in some aspects, first portion 332 may include for example, eighteen indentations 388 (as shown). Alternatively, in other aspects, first portion 332 may include fewer or more indentations 388.

As shown in FIG. 3B, second portion 334 may include a second magnet 374 and a plurality of protrusions 376. Protrusions 376 may be positioned around second magnet 374 in a circular arrangement, for example, around (e.g., spaced apart from) second magnet 374. In some aspects, protrusions 376 are evenly spaced around second magnet 374. In other aspects, protrusions 376 may be unevenly spaced around second magnet 374. In some aspects, as shown, second portion 334 may include four protrusions 376 (as shown). Alternatively, in other aspects, second portion 334 may include fewer or more protrusions 376. For example, in some aspects, second portion 334 may include the same number of protrusions 376 as first portion 332 includes indentations 388.

Protrusions 376 may be sized, shaped, and otherwise configured to be positioned within one of indentations 388, for example, to help secure the position of second portion 334 relative to first portion 332. In some aspects, indentations 388 may be generally circular (e.g., cylindrical or spherical indentations), and protrusions 376 may be generally cylindrical or spherical protrusions. Additionally, second portion 336 may be rotated relative to first portion 332, such that protrusions 376 are repositioned within different indentations 388.

It is noted that the arrangement of coupling device 306 may be reversed. For example, instead of the arrangement shown in FIGS. 3A and 3B, first portion 332 may include protrusions 376, and second portion 334 may include indentations 388. In any of these aspects, protrusions 376 and indentations 388 may help to secure the position of second portion 334 relative to first portion 332, while also helping to allow for relative rotation. In some aspects, second portion 334 includes a circular protrusion 392. Circular protrusion 392 may connect and/or at least partially overlap with each of protrusions 376. For example, protrusions 376 may extend farther from the remainder of second body 352 of second portion 334 by a greater amount or distance than circular protrusion 392. Alternatively, second portion 334 may not include circular protrusion 392, and protrusions 376 may extend from a flat face of second body 352. In any of these aspects, protrusions 376 may at least partially connect or overlap with indentations 388.

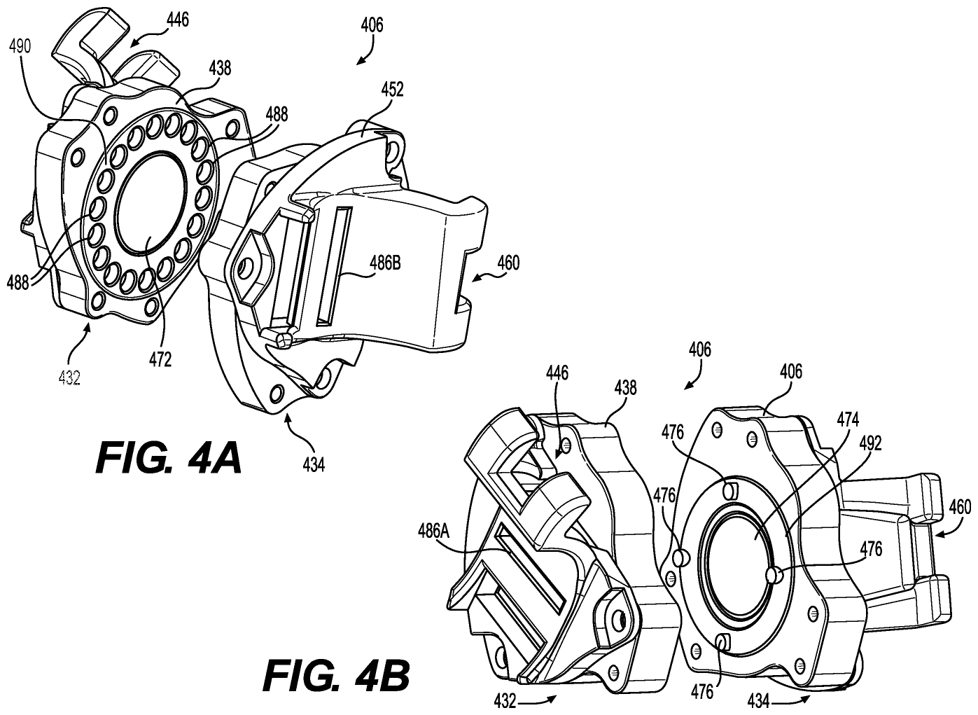

FIGS. 4A and 4B illustrate a perspective view of another coupling device 406, for example, to rotatably couple a first medical device handle and a second medical device handle. Coupling device 406 is similar to coupling device 306, except as noted below. Specifically, coupling device 406 includes a first portion 432 and a second portion 434. First portion 432 includes a first magnet 472 and a plurality of indentations 488, for example, positioned around first magnet 472 in a circular arrangement. Second portion 434 includes a second magnet 474 and a plurality of protrusions 476, with the protrusions configured to be received within one of more of indentations 488. In some aspects, second portion 434 may include a number of protrusions 476 that is fewer than a number of indentations 488 on first portion 432. In these aspects, an amount of force need to rotate second portion 434 relative to first portion 432 when second portion 434 includes a smaller number of protrusions 476 may be less than an amount of force needed to rotate second portion 434 relative to first portion 432 when second portion 434 includes a larger number of protrusions 476. Additionally, second portion 434 may be rotatable relative to first portion 432 more quickly when second portion 434 includes a smaller number of protrusions 476 compared to when second portion 434 includes a larger number of protrusions 476.

Additionally, as shown in FIG. 4A, first portion 432 includes a circular indentation 490, and second portion 434 includes a circular protrusion 492. Circular indentation 490 may connect and/or at least partially overlap with each of indentations 488. For example, indentations 488 may extend inward into first portion 432 from circular indentation 490. That is, circular indentation 490 includes a shallower depth than indentations 488. Moreover, circular protrusion 492 may connect and/or at least partially overlap with each of protrusions 476. For example, protrusions 476 may extend farther from the remainder of second body 452 of second portion 434 by a greater amount or distance than circular protrusion 492. In these aspects, circular indentation 490 and/or circular protrusion 492 may help to maintain a connection between second portion 434 and first portion 432 during the movement of second portion 434 relative to first portion 432.

It is noted that the arrangement of coupling device 406 may be reversed. For example, instead of the arrangement shown in FIGS. 4A and 4B, first portion 432 may include protrusions 476 and circular protrusion 492, and second portion 434 may include indentations 488 and circular indentation 490. In any of these aspects, protrusions 476, indentations 488, circular indentation 490, and circular projection 492 may help to secure the position of second portion 434 relative to first portion 432, while also helping to allow for relative rotation.

It is noted that the size and/or shape of the first and second magnets of coupling devices 206, 306, and 406 may be modified. For example, the size and/or shape of the first and second magnets may be modified to adjust the retention forces caused by the first and second magnets between the first and second portions of coupling devices 206, 306, and 406. In one aspect, the depth and/or radius of the first and second magnets may be increased to increase the retention force between the first and second portions of coupling devices 206, 306, and 406. Alternatively, the depth and/or radius of the first and second magnets may be decreased to decrease the retention force between the first and second portions of coupling devices 206, 306, and 406. Although circular magnets are depicted, it will be appreciated that the magnets may have alternative shapes, and protrusions/indentations of coupling devices 206, 306, 406 may extend around a perimeter thereof or may retain a circular arrangement.

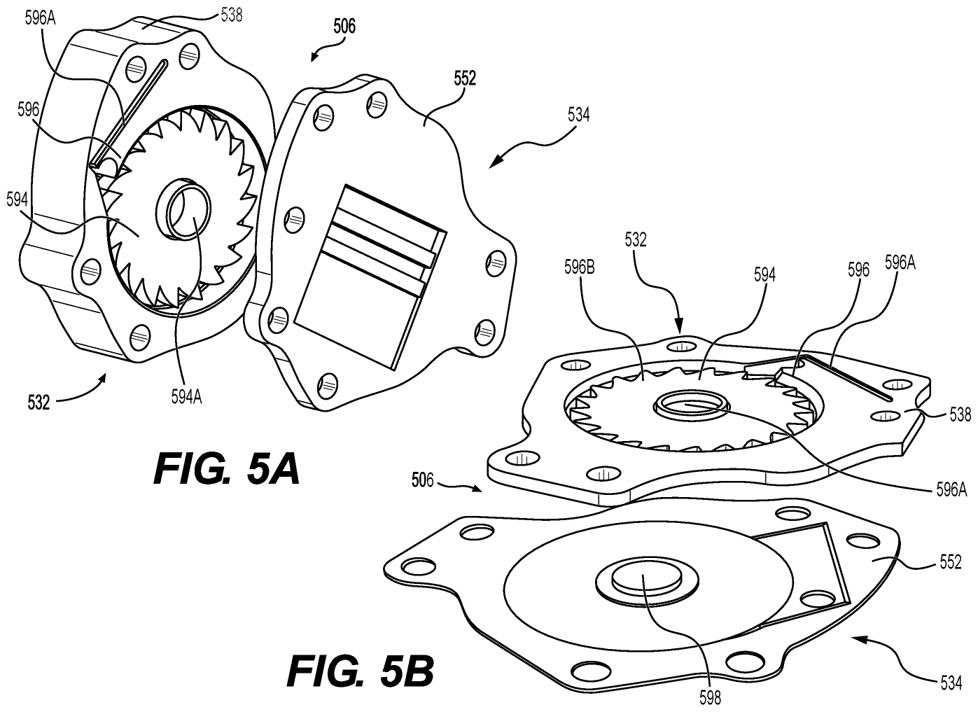

FIGS. 5A and 5B are perspective views of another coupling device 506, for example, to rotatably couple a first medical device handle and a second medical device handle. Coupling device 506 is similar to coupling device 306, except as noted below. Specifically, coupling device 506 includes a first portion 532 and a second portion 534. First portion 532 includes a first body 538, and second portion 534 includes a second body 552. Additionally, as discussed above, the features of first portion 532 and second portion 534 may be reversed.

As shown in FIG. 5A, first portion 532 may include a cogwheel or ratchet 594 and a pawl 596. Ratchet 594 includes a center hole 594A and a plurality of teeth 594B, for example, extending radially outward from ratchet 594. Although not shown, ratchet 594 may be rotatable about an inner ball bearing. Moreover, pawl 596 may be pivotable, for example, via a spring opening 596A. Alternatively, pawl 596 may be formed by a strip of material (e.g., a strip of metal) that may selectively engage with a tooth of ratchet.

Second portion 534 may include a central extension or peg 598 (FIG. 5B), which may be selectively positioned within center hole 594A of ratchet 594. In some aspects, positioning peg 598 in center hole 594A may help to form a pivotable or rotatable snap fit, for example, to couple second portion 534 to first portion 532. Peg 598 may frictionally engage center hole 594A, thereby causing ratchet 594 to rotate as peg 598/second portion 534 rotates.

In these aspects, rotation of second portion 534 may also rotate ratchet 594, for example, clockwise or counterclockwise. However, pawl 596 may limit the rotation of ratchet 594, for example, by interacting with teeth 594B, and thus limit the rotation of second portion 534, by one “click” formed by the next tooth of ratchet 594. In some aspects, pawl 596 may interact with ratchet 594 in order to only allow ratchet 594 to rotate in one direction (e.g., either clockwise or counterclockwise). In these aspects, coupling device 506 may include a release mechanism (not shown) in order to allow rotation in either direction and/or to reset the position of ratchet 594 relative to pawl 596. Alternatively, in other aspects, pawl 596 may interact with ratchet 594 to allow for two-way rotation (e.g., both clockwise and counterclockwise rotation). In any of these aspects, ratchet 594 may include a larger number of teeth 594B (e.g., smaller teeth 594B extending from ratchet 594) in order to provide for finer or smaller incremental adjustments between the relative positions of first portion 532 and second portion 534. Alternatively, ratchet 594 may include a smaller number of teeth 594B (e.g., larger teeth 594B extending from ratchet 594) in order to allow for coarser or larger incremental adjustments between the relative positions of first portion 532 and second portion 534.

Moreover, the interaction of pawl 596 with teeth 594B of ratchet 594 may help to secure the position of second portion 534 relative to first portion 532 unless acted on by the user. The interaction of pawl 596 with teeth 594B of ratchet 594 may also provide an auditory and/or tactile feedback to the user concerning the rotation of second portion 534 relative to first portion 532. Furthermore, first portion 532 and second portion 534 may be uncoupled by uncoupling peg 598 from center hole 594A, for example, by pulling one of first portion 532 or second portion 534 from the other of first portion 532 or second portion 534. Alternatively or additionally, first portion 532 and second portion 534 may be uncoupled by inserting a rod between first portion 532 and second portion 534, for example, to pry first portion 532 and second portion 534 apart.

Although not shown, in some aspects, first portion 532 and second portion 534 may include respective magnets (e.g., similar to first and second magnets discussed above). In one aspect, the magnet for first portion 532 may be positioned within ratchet 594. The magnet for second portion 534 may be positioned adjacent to and/or within peg 598. The magnets may help to maintain a connection between first portion 532 and second portion 534, for example, while first portion 532 and second portion 534 are rotated relative to each other. In these aspects, ratchet 594, center hole 594A, teeth 594B, pawl 596, spring opening 596A, and/or peg 598 may help to secure the position of second portion 534 relative to first portion 532, while also helping to allow for relative rotation.

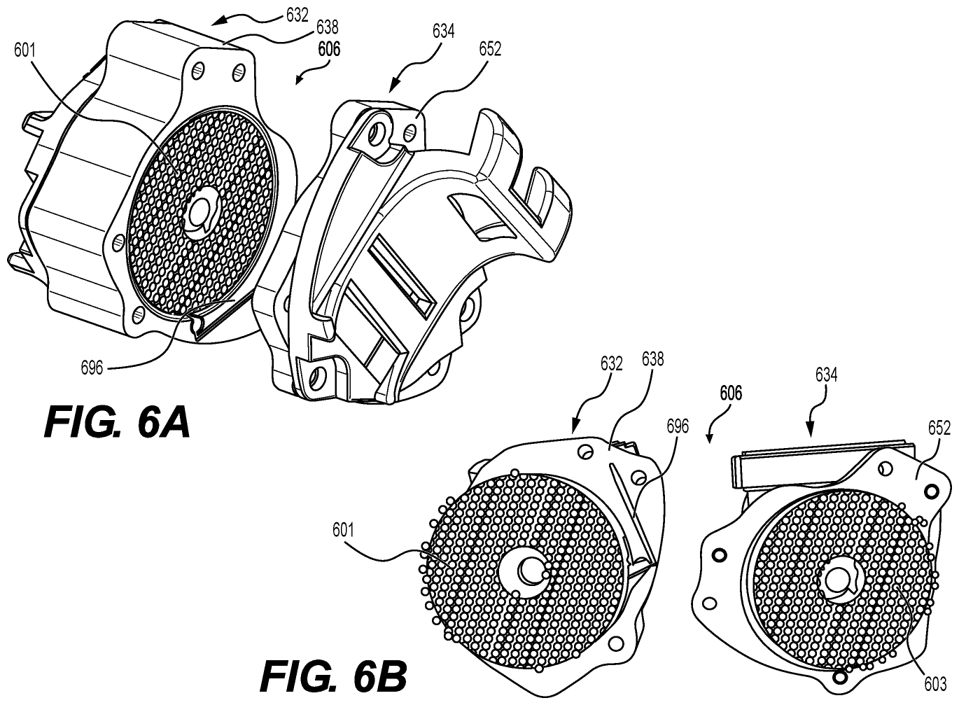

FIGS. 6A and 6B are perspective views of another coupling device 606, for example, to rotatably couple a first medical device handle and a second medical device handle. Coupling device 606 is similar to coupling device 506, except as noted below. Specifically, coupling device 606 includes a first portion 632 and a second portion 634. First portion 632 includes a first body 638, and second portion 634 includes a second body 652.

Coupling device 606 is similar to coupling device 506, except as noted below. For example, first portion 632 may include a ratchet (not shown) and a pawl 696. Although not shown, the ratchet may be rotatable about an inner ball bearing. First portion 632 may include a first Velcro® or hook and loop material 601 (“first material 601”), and second portion 634 may also include a second Velcro® or hook and loop material 603 (“second material 603”). In some aspects, first material 601 is positioned on a face of the ratchet (not shown in FIGS. 6A-6B). First material 601 and second material 603 may be the same material. Alternatively, one of first material 601 or second material 603 may include a plurality of hooks, and the other of first material 601 or second material 603 may include a plurality of loops. In these aspects, first material 601 and second material 603 may help to releasably couple first portion 632 to second portion 634. Alternatively, first portion 632 and second portion 634 may include one or more other materials that may interact to releasably couple first portion 632 and second portion 634.

Rotation of second portion 634 relative to first body 638 may rotate the ratchet relative to pawl 696. As discussed above, pawl 696 may limit the rotation of the ratchet, for example, by interacting with the teeth, and thus limit the rotation of second portion 634, by one “click” formed by the next tooth of the ratchet. Moreover, the interaction of pawl 696 with the teeth of the ratchet may help to secure the position of second portion 634 relative to first portion 632 unless acted on by the user. Furthermore, first portion 632 and second portion 634 may be uncoupled by uncoupling first material 601 and second material 603, for example, by pulling one of first portion 632 or second portion 634 from the other of first portion 632 or second portion 634. Alternatively or additionally, first portion 632 and second portion 634 may be uncoupled by inserting a rod between first portion 632 and second portion 634, for example, to pry first portion 632 and second portion 634 apart.

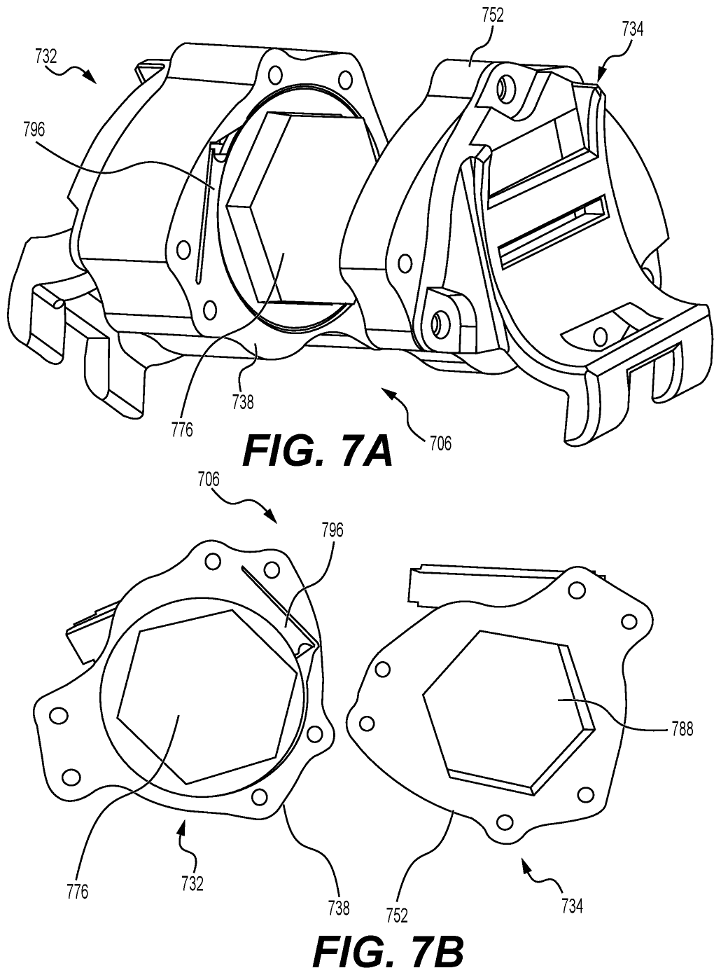

FIGS. 7A and 7B are perspective views of another coupling device 706, for example, to rotatably couple a first medical device handle and a second medical device handle. Specifically, coupling device 706 includes a first portion 732 and a second portion 734. First portion 732 includes a first body 738, and second portion 734 includes a second body 752.

Coupling device 706 is similar to coupling device 506, except as noted below. For example, first portion 732 may include a ratchet (not shown) and a pawl 796. Although not shown, the ratchet may be rotatable about an inner ball bearing. First portion 732 may also include a protrusion 776, and second portion 734 may also include an indentation 788. Protrusion 776 may extend from first body 738, and may form a geometric or polygonal shape. Protrusion 776 may be coupled to the ratchet, such that rotation of protrusion 776 rotates the ratchet. Indentation 788 may extend into second body 752, and may also form a geometric or polygonal shape. For example, protrusion 776 may be a hexagonal shape, as shown. Alternatively, protrusion 776 may be triangular, quadrilateral or rectangular (e.g., square), pentagonal, heptagonal, octagonal, etc. Indentation 788 may be a corresponding shape to protrusion 776, for example, if protrusion 776 is a hexagonal shape, then protrusion 776 may also be a hexagonal shape. In these aspects, first portion 732 and second portion 734 of coupling device 706 may be coupled via a press fit between protrusion 776 and indentation 788. Protrusion 776 may be keyed to indentation 788. Thus, when protrusion 776 is inserted into indentation 788, rotation of second body 752 (and indentation 788) may thereby cause rotation of protrusion 776.

Rotation of second portion 734 relative to first body 738 may rotate the ratchet relative to pawl 796. As discussed above, pawl 796 may limit the rotation of the ratchet, for example, by interacting with the teeth, and thus limit the rotation of second portion 734, by one “click” formed by the next tooth of the ratchet. Moreover, the interaction of pawl 796 with the teeth of the ratchet may help to secure the position of second portion 734 relative to first portion 732 unless acted on by the user. Furthermore, first portion 732 and second portion 734 may be uncoupled by uncoupling protrusion 776 and indentation 788, for example, by pulling one of first portion 732 or second portion 734 from the other of first portion 732 or second portion 734. Alternatively or additionally, first portion 732 and second portion 734 may be uncoupled by inserting a rod between first portion 732 and second portion 734, for example, to pry first portion 732 and second portion 734 apart.

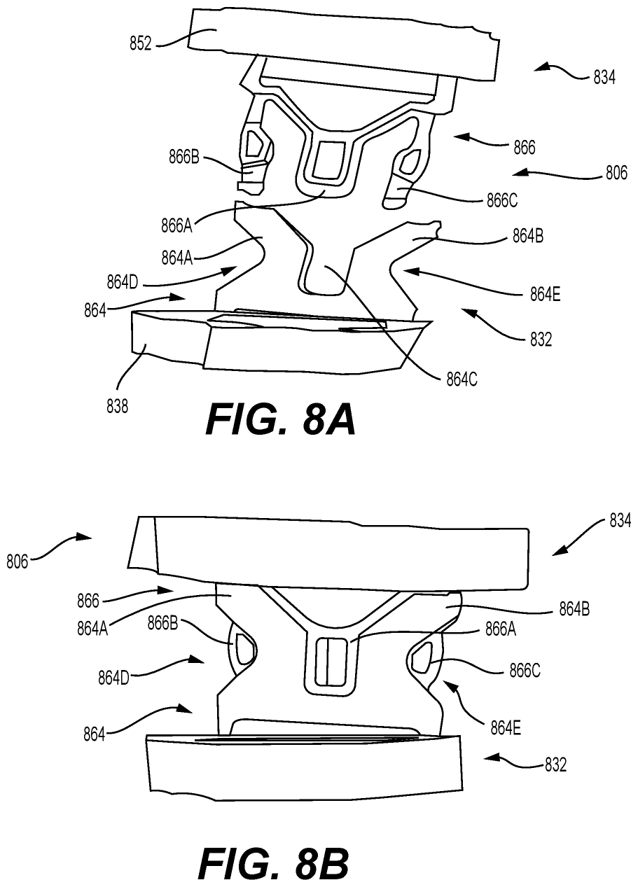

FIGS. 8A and 8B are side views of another coupling device 806, for example, to rotatably couple a first medical device handle and a second medical device handle. Specifically, coupling device 806 includes a first portion 832 and a second portion 834. First portion 832 includes a first body 838, and second portion 834 includes a second body 852. Coupling device 806 is similar to coupling device 306 and coupling device 506, except as noted. For example, although not shown, coupling device 806 may include respective straps, slots, etc. to couple first portion 832 and second portion 834 to respective medical devices.

As shown in FIGS. 8A and 8B, first portion 832 includes a first coupling projection 864, and second portion 834 includes a second coupling projection 866. First coupling projection 864 may include a first extension 864A and a second extension 864B, which may form an opening, for example, a Y-shape opening 864C. Additionally sides (e.g., laterally in FIGS. 8A and 8B) of first extension 864A and second extension 864B may include respective side openings 864D and 864E. Moreover, each of first extension 864A and second extension 864B be hollow or otherwise include internal slots or openings, for example, which may receive portions of second coupling projection 866.

Second coupling projection 866 may include a central prong 866A and one or more side prongs, for example, first side prong 866B and second side prong 866C. First side prong 866B and second side prong 866C may be pivotable, for example, inward toward central prong 866A. As shown in FIG. 8B, when second coupling projection 866 is coupled to first coupling projection 864, central prong 866A may be positioned within opening 864C, for example, to clip second coupling projection 866 into first coupling projection. Additionally, respective portions of first side prong 866B and second side prong 866C may be positioned in the side openings 864D, 864E of first extension 864A and second extension 864B. First portion 832 and second portion 834 may be uncoupled by pushing first side prong 866B and second side prong 866C inward (e.g., toward central prong 866A) and pulling one or more of first portion 832 or second portion 834 away from the other of first portion 832 or second portion 834. Furthermore, it is noted that the features of first portion 832 and second portion 834 may be reversed, for example, such that first portion 832 includes prongs 866A-866C and second portion 834 includes extensions 864A and 864B.

Moreover, first coupling projection 864 may be rotatable relative to first body 838. Alternatively, although not shown, second coupling projection 866 may be rotatable relative to second body 852. In these aspects, one or more of first portion 832 or second portion 834 may include a ratchet and pawl mechanism, for example, similar to the ratchet and pawl mechanisms and/or ball bearings discussed above.

Various aspects discussed herein may help reduce procedure time, increase tissue treatment effectiveness, reduce the risks to the subject, etc. Various aspects of this disclosure may allow for one or more users to use multiple endoscopic instruments for the resection of tissue, or otherwise provide treatment to the subject. In some instances, as discussed herein, a secondary medical device may be coupled to and/or inserted through a portion of a primary medical device in order allow one user to hold and/or manipulate both the primary medical device and the secondary medical device, for example, to treat a treatment site. Additionally, as discussed herein, coupling the secondary medical device to the primary medical device and/or inserting the secondary medical device through the primary device may not limit the movement of the secondary medical device relative to primary medical device. As a result, the user may experience less fatigue, may experience a larger range of motion, etc. Additionally, the secondary medical device may be uncoupled from the primary medical device, for example, for operation by another user, and may later be re-coupled to the primary medical device. Alternatively or additionally, the secondary medical device may be uncoupled from the primary medical device, and another secondary medical device may be coupled to the primary medical device and operated by the user.

It also should be understood that one or more aspects of any of the medical devices, systems, and methods described herein may be used for grasping, cutting, dissecting, treating, ablating, or otherwise manipulating tissue in any part of the human body. For example, any of the medical devices described herein may be used in medical procedures such as for Endoscopic Submucosal Dissection (ESD), cancer treatment, kidney or bladder biopsies or resections, and/or other procedures where removal, resection, dissection, fulguration, and/or ablation of tissue is needed.

Although the exemplary embodiments described above have been disclosed in connection with medical devices for manipulating and cutting human tissue through one or more working channels of a medical device, a natural orifice, or by incision, a person skilled in the art will understand that the principles set out above can be applied to any medical device or medical method and can be implemented in different ways without departing from the scope of the disclosure as defined by the claims. In particular, constructional details, including manufacturing techniques and materials, are well within the understanding of those of skill in the art and have not been set out in any detail here. These and other modifications and variations are well within the scope of this disclosure and can be envisioned and implemented by those of skill in the art.

Moreover, while specific exemplary embodiments may have been illustrated and described collectively herein, it should be appreciated that any subsequent arrangement designed to achieve the same or similar purpose may be substituted for the specific embodiments described and shown herein. This disclosure is intended to cover any and all subsequent adaptations or variations of various embodiments. Combinations of the above embodiments, and other embodiments not specifically described herein, will be apparent to those of skill in the art upon reviewing the description.

While principles of the disclosure are described herein with reference to illustrative aspects for particular applications, it should be understood that the disclosure is not limited thereto. Those having ordinary skill in the art and access to the teachings provided herein will recognize additional modifications, applications, aspects, and substitution of equivalents all fall within the scope of the aspects described herein. Accordingly, the disclosure is not to be considered as limited by the foregoing description.

Other exemplary embodiments of this disclosure will be apparent to those skilled in the art from consideration of the specification and practice of the exemplary embodiments disclosed herein. It is intended that the specification and examples be considered as exemplary only, and departures in form and detail may be made without departing from the scope and spirit of this disclosure as defined by the following claims.

We claim:

1. A coupling device for a medical system, comprising:
a first portion, wherein the first portion includes a first body and a first connector, wherein the first connector extends from the first body and is configured to couple a first medical device to the first portion; and
a second portion, wherein the second portion includes a second body and a second connector, wherein the second connector extends from the second body and is configured to couple a second medical device to the second portion,
wherein the first portion and the second portion are removably couplable, wherein, when coupled, the second portion is rotatable relative to the first portion.

2. The coupling device of claim 1, wherein the first body includes a first coupling projection, and wherein the second body includes a second coupling projection configured to be coupled to the first coupling projection.

3. The coupling device of claim 2, wherein the first coupling projection includes a socket, and wherein the second coupling projection includes a ball.

4. The coupling device of claim 1, wherein the first body includes a first magnet, and wherein the second body includes a second magnet of opposite polarity to the first magnet.

5. The coupling device of claim 4, wherein the first body includes a plurality of first protrusions, and wherein the second body includes a plurality of second protrusions configured to abut and rotate relative to the first protrusions.

6. The coupling device of claim 5, wherein each of the first protrusions and the second protrusions includes a trapezoidal shape with angled side surfaces.

7. The coupling device of claim 5, wherein the first body further includes a circular depression, wherein the first protrusions are positioned within the circular depression.

8. The coupling device of claim 4, wherein the first body includes a plurality of indentations positioned in a circular arrangement around the first magnet, and wherein the second body includes a plurality of protrusions in a circular arrangement around the second magnet.

9. The coupling device of claim 8, wherein the indentations are generally circular, and wherein the protrusions are generally circular, wherein the first body further includes a circular indentation that at least partially overlaps with the indentations, and wherein the second body further includes a circular protrusion that at least partially overlaps with the protrusions.

10. The coupling device of claim 1, wherein the first body includes a ratchet and a pawl.

11. The coupling device of claim 10, wherein the ratchet includes a center hole, and wherein the second body includes a peg configured to positioned within the center hole to couple the second body to the first body.

12. The coupling device of claim 10, wherein the first body includes a first material, and wherein the second body includes a second material that is releasably couplable to the first material.

13. The coupling device of claim 1, wherein the first body includes a protrusion, and wherein the second body includes an indentation configured to key with and be coupled to the protrusion via a press fit.

14. The coupling device of claim 1, wherein the first body includes a first coupling projection with first and second extensions that each include respective sides openings, and
wherein the second body includes a second coupling projection with a central prong, a first side prong, and a second side prong, wherein the first side prong and the second side prong are both pivotable inward toward the central prong to couple or uncouple the first body and the second body.

15. The coupling device of claim 1, wherein the first body includes a first receiving portion with a first slot, and wherein the first connector includes a first narrowed portion configured to be positioned within the first slot,
wherein the second body includes a second receiving portion with a second slot, and wherein the second connector includes a second narrowed portion configured to be positioned within the second slot.

16. A medical system, comprising:
a first medical device, including a first handle and a first insertion portion, wherein the first handle includes a port;
a second medical device, including a second handle and a second insertion portion, wherein at least a portion of the second insertion portion is configured to be inserted into the port; and
a coupling device, wherein the coupling device includes:
a first portion, wherein the first portion includes a first body, wherein the first body includes a first receiving portion with a first slot, and wherein the first slot is configured to receive a portion of a first strap to couple the first handle to the first portion; and
a second portion, wherein the second portion includes a second body, wherein the second body includes a second receiving portion with a second slot, and wherein the second slot is configured to receive a portion of a second strap to couple the second handle to the second portion,
wherein the first portion and the second portion are removably couplable, wherein, when coupled, the second portion is rotatable relative to the first portion such that the second handle is rotatable relative to the first handle.

17. The medical system of claim 16, wherein the first body includes a first coupling projection, wherein the second body includes a second coupling projection configured to be coupled to the first coupling projection, wherein the first coupling projection includes a socket, and wherein the second coupling projection includes a ball.

18. The medical system of claim 16, wherein the first body includes a first magnet, wherein the second body includes a second magnet of opposite polarity to the first magnet,
wherein the first body includes a plurality of first protrusions, wherein the second body includes a plurality of second protrusions configured to abut and rotate relative to the first protrusions, and wherein each of the first protrusions and the second protrusions includes a trapezoidal shape with angled side surfaces.

19. A coupling device, comprising:
a first portion, wherein the first portion includes a first body and a first strap, wherein the first strap extends from the first body and is configured to couple a first medical device to the first portion, and wherein the first portion includes a socket extending from the first body; and
a second portion, wherein the second portion includes a second body and a second strap, wherein the second strap extends from the second body and is configured to couple a second medical device to the second portion, and wherein the second portion includes a hollow ball configured to be positioned within the socket of the first portion,
wherein the first portion and the second portion are removably couplable, wherein, when coupled, the second portion is pivotable relative to the first portion through a semi-spherical range of motion.

20. The coupling device of claim 19, wherein the first body includes a first receiving portion with a first slot, and wherein the first strap includes a first narrowed portion configured to be positioned within the first slot,
wherein the second body includes a second receiving portion with a second slot, and wherein the second strap includes a second narrowed portion configured to be positioned within the second slot.
